(19) United States
(12) Patent Application Publication
(10) Pub. No.: US 2010/0185096 A1
(43) Pub. Date: Jul. 22, 2010

(54) Ultrasonic probe and ultrasonic diagnostic apparatus

(75) Inventors: Yukiya Miyachi, Kaisei-machi (JP); Tomohiro Nishino, Kaisei-machi (JP); Yukio Sugita, Kaisei-machi (JP)

Correspondence Address: AKERMAN SENTERFITT, 8100 BOONE BOULEVARD, SUITE 700, VIENNA, VA, 22182-2683, US

(73) Assignee: FUJIFILM Corporation, Tokyo (JP)

(21) Appl. No.: 12/654,958

(22) Filed: Jan. 11, 2010

(30) Foreign Application Priority Data
Jan. 16, 2009 (JP) 2009-007420
Mar. 31, 2009 (JP) 2009-085263

(51) Int. Cl. A61B 8/14

(52) U.S. Cl. 600/459

(57) ABSTRACT

Power consumption is suppressed when received power is insufficient for wireless power feed to an ultrasonic probe. The ultrasonic probe includes: plural ultrasonic transducers for transmitting and receiving ultrasonic waves; a signal processing unit for performing signal processing on reception signals outputted from the plural ultrasonic transducers to generate a transfer signal; an energy conversion unit for converting energy wirelessly fed from a power feeding device into electric energy; a power receiving status detecting unit for detecting an amount of the energy wirelessly fed from the power feeding device, and determining whether or not the ultrasonic probe is within a region where the energy wirelessly fed from the power feeding device can be received; and a transmitting unit for transmitting a determination result of the power receiving status detecting unit to the power feeding device.

### CROSS-REFERENCE TO RELATED APPLICATION

The present application claims priority from Japanese Patent Applications No. 2009-007420 filed on Jan. 16, 2009 and No. 2009-085263 filed on Mar. 31, 2009, the contents of which are incorporated herein by reference in their entirety.

### BACKGROUND OF THE INVENTION

1. Field of the Invention

The present invention relates to an ultrasonic probe including plural ultrasonic transducers for transmitting and receiving ultrasonic waves, and an ultrasonic diagnostic apparatus including the ultrasonic probe and an ultrasonic diagnostic apparatus main body.

2. Description of a Related Art

In medical fields, various imaging technologies have been developed for observation and diagnoses within an object to be inspected. Especially, ultrasonic imaging for acquiring interior information of the object by transmitting and receiving ultrasonic waves enables image observation in real time and provides no exposure to radiation unlike other medical image technologies such as X-ray photography or RI (radio isotope) scintillation camera. Accordingly, ultrasonic imaging is utilized as an imaging technology at a high level of safety in a wide range of departments including not only the fetal diagnosis in obstetrics, but also gynecology, circulatory system, digestive system, and so on.

The principle of ultrasonic imaging is as follows. Ultrasonic waves are reflected at a boundary between regions having different acoustic impedances like a boundary between structures within the object. Therefore, by transmitting ultrasonic beams into the object such as a human body and receiving ultrasonic echoes generated within the object, and obtaining reflection points, where the ultrasonic echoes are generated, and reflection intensity, outlines of structures (e.g., internal organs, diseased tissues, and so on) existing within the object can be extracted.

Generally, in an ultrasonic diagnostic apparatus, an ultrasonic probe including plural ultrasonic transducers (vibrators) having transmitting and receiving functions of ultrasonic waves is used. The ultrasonic probe and an ultrasonic diagnostic apparatus main body are often connected via a cable. However, in order to remove the burden of using the cable, ultrasonic diagnostic apparatuses of a wireless communication type for performing wireless information communication between the ultrasonic probe and the ultrasonic diagnostic apparatus main body are being developed.

In some ultrasonic diagnostic apparatuses of the wireless communication type, a secondary battery is built in the ultrasonic probe and appropriately charged for use. Further, in order to prevent contact failure or electric leakage, a technology of charging a secondary battery by wireless power feed using electromagnetic induction without exposure of electric contacts is proposed.

As a related technology, Japanese Patent Application Publication JP-P2003-10177A discloses an ultrasonic diagnostic apparatus including an ultrasonic probe having power receiving means for receiving power by electromagnetic induction and charging means for charging a secondary battery with the power received by the receiving means, but having no exposed electric contact, and an ultrasonic diagnostic apparatus main body having power feeding means for feeding power by electromagnetic induction. In the ultrasonic diagnostic apparatus, by inserting the ultrasonic probe into, for example, a probe receiver of the ultrasonic diagnostic apparatus main body, the power feeding means and the charging means are closely positioned for higher power feed efficiency.

However, depending on the location of the ultrasonic probe, the power feed efficiency is low and the ultrasonic probe cannot sufficiently receive the power. If an attempt to feed power to the ultrasonic probe is made when the ultrasonic probe cannot sufficiently receive the power, there is a problem of wasted power consumption in the power feeding means. Further, how much time the ultrasonic probe is available is unknown, and there is a problem of poor usability.

### SUMMARY OF THE INVENTION

The present invention has been achieved in view of the above-mentioned problems. A first purpose of the present invention is to provide an ultrasonic probe and an ultrasonic diagnostic apparatus that can suppress power consumption when received power is insufficient for wireless power feed to the ultrasonic probe. Further, a second purpose of the present invention is to provide an ultrasonic probe and an ultrasonic diagnostic apparatus that can determine how much time the ultrasonic probe is available so as to improve usability.

In order to accomplish the above-mentioned purposes, an ultrasonic probe according to a first aspect of the present invention includes: plural ultrasonic transducers for transmitting ultrasonic waves according to drive signals, and receiving ultrasonic echoes to output reception signals; a signal processing unit for performing signal processing on the reception signals outputted from the plural ultrasonic transducers to generate a transfer signal; an energy conversion unit for converting energy wirelessly fed from a power feeding device into electric energy; a power receiving status detecting unit for detecting an amount of the energy wirelessly fed from the power feeding device, and determining whether or not the ultrasonic probe is within a region where the energy wirelessly fed from the power feeding device can be received; and a transmitting unit for transmitting a determination result of the power receiving status detecting unit to the power feeding device.

Further, an ultrasonic probe according to a second aspect of the present invention includes: plural ultrasonic transducers for transmitting ultrasonic waves according to drive signals, and receiving ultrasonic echoes to output reception signals; a signal processing unit for performing signal processing on the reception signals outputted from the plural ultrasonic transducers to generate a transfer signal; an energy conversion unit for converting energy wirelessly fed from a power feeding device into electric energy; a battery for accumulating the electric energy converted by the energy conversion unit and supplying electric power to at least the signal processing unit; a remaining battery charge detecting unit for detecting remaining battery charge of the battery; and a feed time determining unit for determining a time, in which electric power can be supplied from the battery, based on the remaining battery charge detected by the remaining battery charge detecting unit.

According to the first aspect of the present invention, since the ultrasonic probe is provided with the power receiving status detecting unit for determining whether or not the ultrasonic probe is within the region where the energy wirelessly fed from the power feeding device can be received, the power consumption can be suppressed when received power is insufficient for wireless power feed to the ultrasonic probe.

Further, according to the second aspect of the present invention, since the ultrasonic probe is provided with the feed time determining unit for determining the time, in which electric power can be supplied from the battery, based on the remaining battery charge detected by the remaining battery charge detecting unit, how much time the ultrasonic probe is available can be determined so as to improve usability.

### BRIEF DESCRIPTION OF THE DRAWINGS

FIG. 1 is a perspective view showing a schematic configuration of an ultrasonic diagnostic apparatus according to embodiments of the present invention;

FIG. 2 is a block diagram showing a configuration of an ultrasonic probe according to the first embodiment of the present invention;

FIG. 3 is a block diagram showing a configuration of an ultrasonic diagnostic apparatus main body according to the first embodiment of the present invention;

FIG. 4 shows a configuration example of a reception signal processing unit as shown in FIG. 2;

FIG. 5 is a circuit diagram showing a configuration example of a power receiving unit and a received power detecting unit as shown in FIG. 2;

FIG. 6 is a flowchart for explanation of an operation example of the ultrasonic diagnostic apparatus according to the first embodiment of the present invention;

FIG. 7 is a block diagram showing a configuration of an ultrasonic probe according to the second embodiment of the present invention;

FIG. 8 is a block diagram showing a configuration of an ultrasonic diagnostic apparatus main body according to the second embodiment of the present invention;

FIG. 9 is a graph showing a principle of computing a remaining time in which electric power can be supplied from a battery; and

FIG. 10 is a flowchart for explanation of an operation example of an ultrasonic diagnostic apparatus according to the second embodiment of the present invention.

### DESCRIPTION OF THE PREFERRED EMBODIMENTS

Hereinafter, embodiments of the present invention will be explained in detail with reference to the drawings. The same reference characters are assigned to the same component elements and the explanation thereof will be omitted.

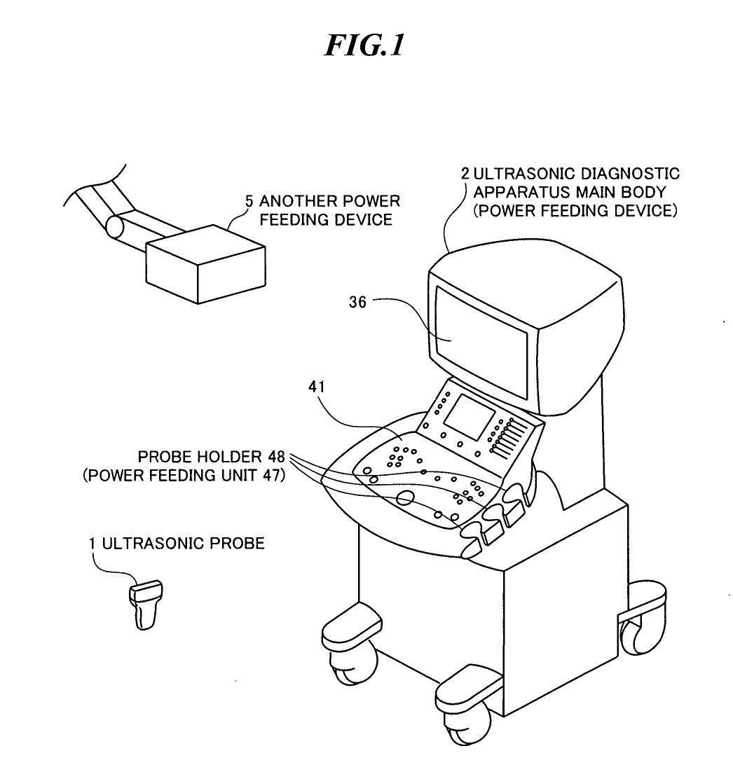

FIG. 1 is a perspective view showing a schematic configuration of an ultrasonic diagnostic apparatus according to embodiments of the present invention. The ultrasonic diagnostic apparatus according to the embodiments of the present invention includes an ultrasonic probe 1 and an ultrasonic diagnostic apparatus main body 2. The ultrasonic diagnostic apparatus main body 2 includes a power feeding unit 47, which will be described later, enabling wireless power feed to the ultrasonic probe 1. Therefore, the ultrasonic diagnostic apparatus main body 2 also has a function of a power feeding device. Further, a power feeding device 5 other than the ultrasonic diagnostic apparatus main body 2 may wirelessly feed power to the ultrasonic probe 1.

First, an ultrasonic diagnostic apparatus according to the first embodiment of the present invention will be explained.

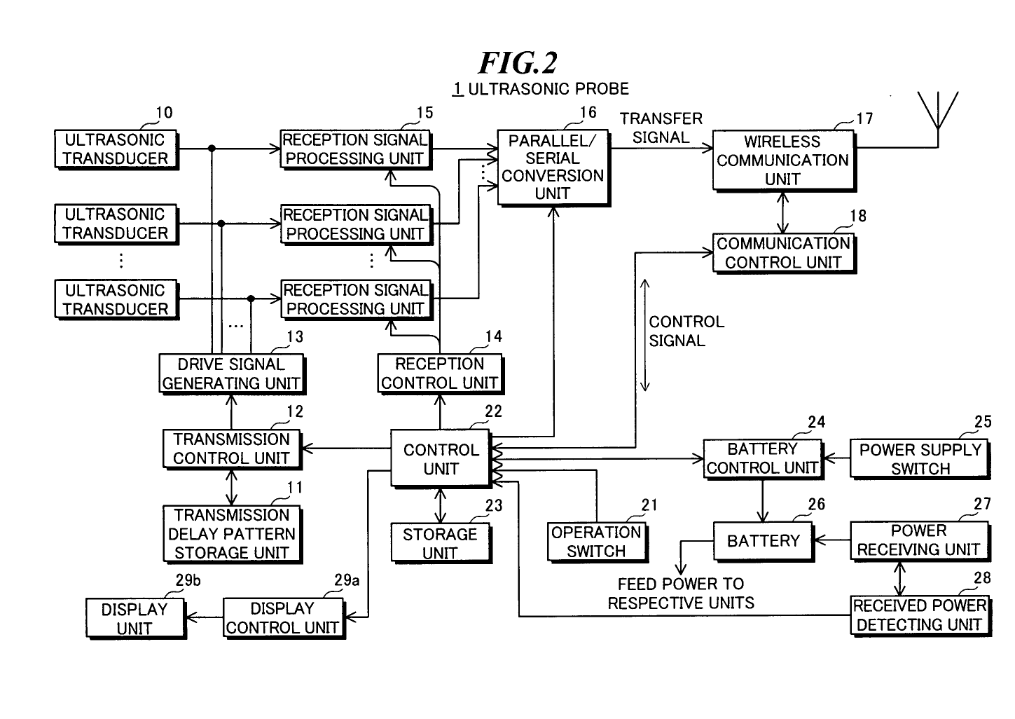

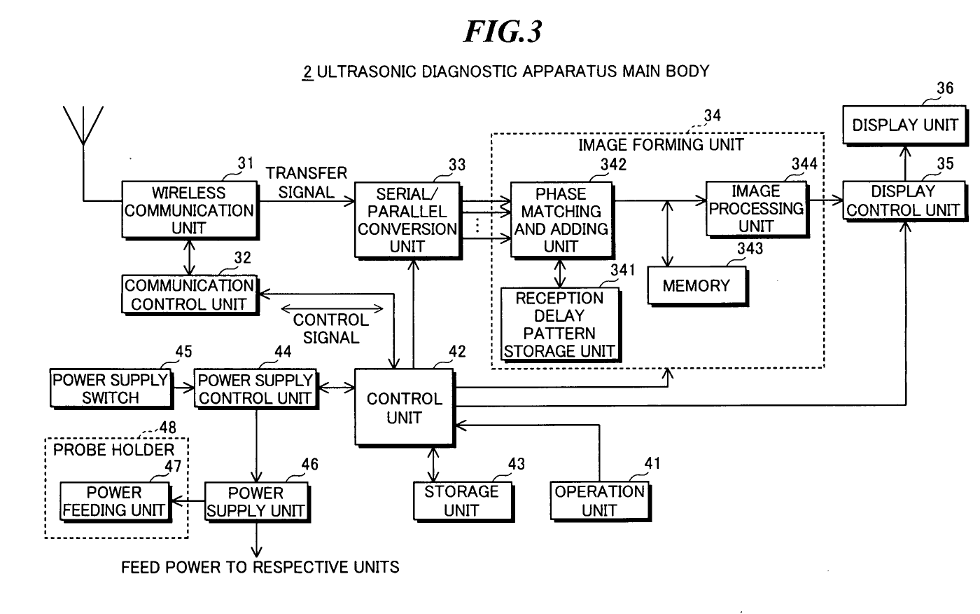

FIG. 2 is a block diagram showing a configuration of an ultrasonic probe according to the first embodiment of the present invention, and FIG. 3 is a block diagram showing a configuration of an ultrasonic diagnostic apparatus main body according to the first embodiment of the present invention. The ultrasonic probe 1 may be an external probe of linear-scan type, convex-scan type, sector-scan type, or the like, or an ultrasonic endoscopic probe of radial-scan type or the like.

As shown in FIG. 2, the ultrasonic probe 1 includes plural ultrasonic transducers 10 forming a one-dimensional or two-dimensional transducer array, a transmission delay pattern storage unit 11, a transmission control unit 12, a drive signal generating unit 13, a reception control unit 14, plural channels of reception signal processing units 15, a parallel/serial conversion unit 16, a wireless communication unit 17, a communication control unit 18, an operation switch 21, a control unit 22, a storage unit 23, a battery control unit 24, a power supply switch 25, a battery 26, a power receiving unit 27, a received power detecting unit 28, a display control unit 29a, and a display unit 29b.

Here, the reception signal processing units 15 and the parallel/serial conversion unit 16 form a signal processing unit for performing signal processing on reception signals outputted from the plural ultrasonic transducers 10 to generate a transfer signal. The power receiving unit 27 forms an energy conversion unit for converting energy wirelessly fed from the power feeding device into electric energy. The received power detecting unit 28 and the control unit 22 form a power receiving status detecting unit for detecting an amount of the energy wirelessly fed from the power feeding device, and determining whether or not the ultrasonic probe is within a region where the energy wirelessly fed from the power feeding device can be received. The wireless communication unit 17 and the communication control unit 18 form a transmitting unit for transmitting the transfer signal generated by the signal processing unit and a detection signal representing a detection result of the received power detecting unit 28 and/or a determination result of the control unit 22 to the power feeding device. And, the battery control unit 24 forms a power supply selecting unit for selecting a battery as a drive power supply in the case where the power receiving status detecting unit determines that the ultrasonic probe is not within the region where the supplied energy can be received.

The plural ultrasonic transducers 10 transmit ultrasonic waves according to applied drive signals, and receive propagating ultrasonic echoes to output reception signals. Each ultrasonic transducer 10 includes a vibrator having electrodes formed on both ends of a material having a piezoelectric property (piezoelectric material) such as a piezoelectric ceramic represented by PZT (Pb (lead) zirconate titanate), a polymeric piezoelectric element represented by PVDF (polyvinylidene difluoride), or the like.

When a pulsed or continuous wave voltage is applied to the electrodes of the vibrator, the piezoelectric material expands and contracts. By the expansion and contraction, pulse or continuous wave ultrasonic waves are generated from the respective vibrators, and an ultrasonic beam is formed by synthesizing these ultrasonic waves. Further, the respective vibrators expand and contract by receiving the propagating ultrasonic waves and generate electric signals. These electric signals are outputted as reception signals of ultrasonic waves.

The transmission delay pattern storage unit 11 stores plural transmission delay patterns to be used when an ultrasonic beam is formed by using ultrasonic waves transmitted from the plural ultrasonic transducers 10. The transmission control unit 12 selects one transmission delay pattern from among the plural transmission delay patterns stored in the transmission delay pattern storage unit 11 according to a transmission direction set by the control unit 22, and sets delay times to be respectively provided to the drive signals for the plural ultrasonic transducers 10 based on the selected transmission delay pattern. Alternatively, the transmission control unit 12 may set the delay times such that the ultrasonic waves transmitted at a time from the plural ultrasonic transducers 10 reach the entire imaging region of the object.

The drive signal generating unit 13 includes plural pulsers, for example, and adjusts the amounts of delay of the drive signals such that the ultrasonic waves transmitted from the plural ultrasonic transducers 10 form an ultrasonic beam and supplies the drive signals to the plural ultrasonic transducers 10, or supplies the drive signals to the plural ultrasonic transducers 10 such that the ultrasonic waves transmitted at a time from the plural ultrasonic transducers 10 reach the entire imaging region of the object.

The reception control unit 14 controls the operation of the plural channels of reception signal processing units 15. Each channel of reception signal processing unit 15 performs orthogonal detection processing or orthogonal sampling processing on the reception signal outputted from a respective one of the ultrasonic transducers 10 to generate a complex baseband signal, and samples the complex baseband signal to generate sample data, and then, supplies the sample data to the parallel/serial conversion unit 16.

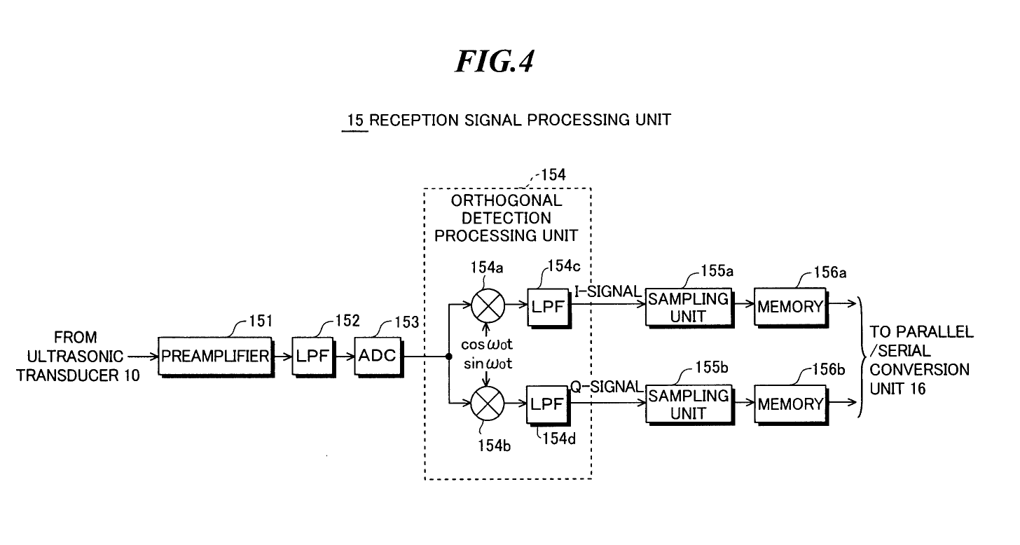

FIG. 4 shows a configuration example of the reception signal processing unit as shown in FIG. 2. As shown in FIG. 4, each channel of reception signal processing unit 15 includes a preamplifier 151, a low-pass filter (LPF) 152, an analog/digital converter (ADC) 153, an orthogonal detection processing unit 154, sampling units 155a and 155b, and memories 156a and 156b.

The preamplifier 151 amplifies the reception signal (RF signal) outputted from the ultrasonic transducer 10, and the LPF 152 limits a frequency band of the reception signal outputted from the preamplifier 151 to prevent aliasing in A/D conversion. The ADC 153 converts the analog reception signal outputted from the LPF 152 into a digital reception signal.

If serialization of data remaining in the RF signals is performed, the transmission bit rate becomes extremely higher and the communication speed and the operation speed of the memories cannot keep up with the bit rate. On the other hand, if the data is serialized after reception focusing processing, the transmission bit rate can be reduced. However, a circuit for reception focusing processing is large-scaled and hard to be incorporated into the ultrasonic probe. Accordingly, in the embodiment, orthogonal detection processing or the like is performed on the reception signal to convert the frequency range of the reception signal into the baseband frequency range and then the data is serialized so that the transmission bit rate is reduced.

The orthogonal detection processing unit 154 performs orthogonal detection processing on the reception signal to generate a complex baseband signal (I-signal and Q-signal). As shown in FIG. 4, the orthogonal detection processing unit 154 includes mixers (multiplication circuits) 154a and 154b, and low-pass filters (LPFs) 154c and 154d. The mixer 154a multiplies the reception signal by a local oscillation signal cosw₀t, the LPF 154c performs low-pass filter processing on the signal outputted from the mixer 154a, and thereby, an I-signal representing a real number component of the complex baseband signal is generated. On the other hand, the mixer 154b multiplies the reception signal by a local oscillation signal sinw₀t, which is obtained by shifting the phase of the local oscillation signal cosw₀t by p/2, the LPF 154d performs low-pass filter processing on the signal outputted from the mixer 154b, and thereby, a Q-signal representing an imaginary number component of the complex baseband signal is generated.

The sampling units 155a and 155b sample (resample) the complex baseband signal (I-signal and Q-signal) generated by the orthogonal detection processing unit 154. Thereby, two channels of sample data are generated. The generated two channels of sample data are stored in the memories 156a and 156b, respectively.

Referring to FIG. 2 again, the parallel/serial conversion unit 16 converts the parallel sample data generated by the plural channels of reception signal processing units 15 into serial sample data (transfer signal). For example, the parallel/serial conversion unit 16 converts 128 channels of parallel data obtained based on the 64 reception signals outputted from the 64 ultrasonic transducers into one channel or two, three or four channels of serial sample data. Thereby, compared to the number of ultrasonic transducers 10, the number of transmission channels is significantly reduced.

The wireless communication unit 17 modulates a carrier signal based on the transfer signal to generate a transmission signal, and supplies the transmission signal to an antenna to transmit electric waves from the antenna, and thereby, transmits a transfer signal. As a modulation system, for example, ASK (Amplitude Shift Keying), PSK (Phase Shift Keying), QPSK (Quadrature Phase Shift Keying), 16QAM (16 Quadrature Amplitude Modulation), or the like is used. In the case of using ASK or PSk, one channel of serial data can be transmitted in one route, in the case of using QPSK, two channels of serial data can be transmitted in one route, and in the case of using 16QAM, four channels of serial data can be transmitted in one route.

Further, the wireless communication unit 17 modulates a carrier signal based on a detection signal representing a detection result of the received power detecting unit 28 to generate a transmission signal, and supplies the transmission signal to the antenna to transmit electric waves from the antenna, and thereby, transmits the detection signal.

In this manner, the wireless communication unit 17 performs wireless communication between the ultrasonic diagnostic apparatus main body 2 and itself, and thereby, transmits the transfer signal and the detection signal to the ultrasonic diagnostic apparatus main body 2, and receives various kinds of control signals from the ultrasonic diagnostic apparatus main body 2 to output the received control signals to the communication control unit 18. The communication control unit 18 controls the wireless communication unit 17 such that the transfer signal and the detection signal are transmitted with transmission electric wave intensity set by the control unit 22, and outputs the various kinds of control signals received by the wireless communication unit 17 to the control unit 22. The control unit 22 controls the respective units of the ultrasonic probe 1 according to the various kinds of control signals transmitted from the ultrasonic diagnostic apparatus main body 2.

The operation switch 21 includes a switch for setting the ultrasonic diagnostic apparatus in a live mode or a freeze mode. Here, the live mode is a mode of displaying a moving image based on the reception signals sequentially obtained by transmitting and receiving ultrasonic waves, and the freeze mode is a mode of displaying a still image based on the reception signals or sound ray signals stored in the memory or the like. The setting signal for the live mode or the freeze mode is included in the transmission signal together with the transfer signal, and transmitted to the ultrasonic diagnostic apparatus main body 2. In addition, the switching between the live mode and the freeze mode may be performed in the ultrasonic diagnostic apparatus main body 2.

The battery 26 supplies electric power to the respective units, which require power, such as the drive signal generating unit 13, the reception signal processing units 15, the parallel/serial conversion unit 16, the wireless communication unit 17, the control unit 22, and so on. The ultrasonic probe 1 is provided with the power supply switch 25, and the battery control unit 24 controls whether or not the power is supplied to the respective units from the battery 26 according to the status of the power supply switch 25. The battery 26 can be charged by using electric energy obtained by the power receiving unit 27 from the fed energy.

The power receiving unit 27 is an electric circuit for converting the energy wirelessly fed from the power feeding unit 47 of the ultrasonic diagnostic apparatus main body 2 (FIG. 3) or the other power feeding device 5 (FIG. 1) into electric energy, and thereby, receives wirelessly fed power. The power receiving unit 27 uses an LC resonance circuit, for example, to generate an induced electromotive force from a magnetic field generated by the power feeding unit 47 or the like, and rectifies the induced electromotive force to convert it into a predetermined direct-current voltage. In FIG. 2, the output terminal of the power receiving unit 27 is connected to the battery 26 and electric power is supplied from the battery 26 to the respective units of the ultrasonic probe 1, but the power receiving unit 27 may be a direct power supply connected to the respective units of the ultrasonic probe 1 not via the battery 26. In this case, selection of the battery 26 or the power receiving unit 27 as the power supply for the respective units of the ultrasonic probe 1 is performed by the battery control unit 24.

The received power detecting unit 28 is an electric circuit for detecting whether or not power is received by the power receiving unit 27 from the power feeding unit 47 and/or intensity of the power (an amount of energy wirelessly fed from the power feeding device). The received power detecting unit 28 includes a current measurement circuit for measuring a current by the induced electromotive force generated in the power receiving unit 27, as will be explained below.

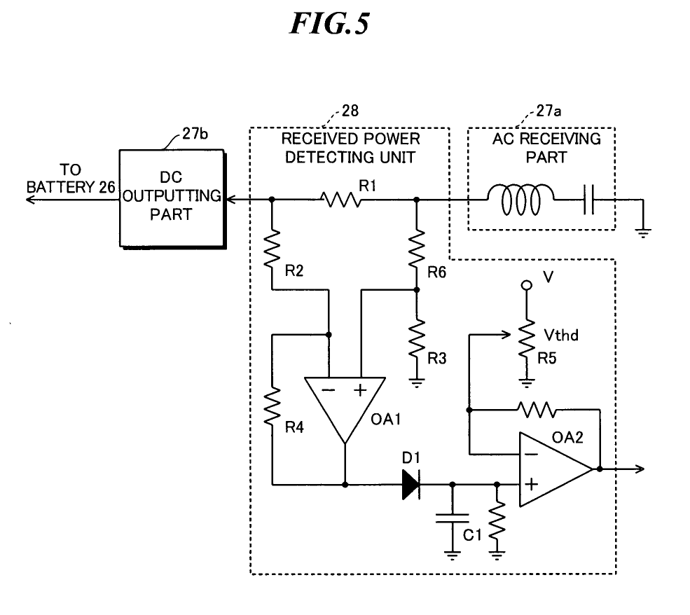

FIG. 5 is a circuit diagram showing a configuration example of the power receiving unit and the received power detecting unit as shown in FIG. 2. The power receiving unit 27 as shown in FIG. 5 includes an AC receiving part 27a for generating an induced electromotive force from a magnetic field generated by the power feeding unit 47 or the like, and a DC outputting part 27b for rectifying and converting the induced electromotive force into a predetermined direct-current voltage. As shown in FIG. 5, the received power detecting unit 28 includes a resistor R1 that is connected in series between the AC receiving part 27a and the DC outputting part 27b. The both ends of the resistor R1 are connected to a non-inverting input terminal and an inverting input terminal of an operational amplifier OA1 via resistors R6 and R2, respectively. Therefore, the potential difference between the both ends of the resistor R1, which is determined by a current value generated in the power receiving unit 27 and a resistance value of the resistor R1, is differential-amplified by the operational amplifier OA1. The output signal of the operational amplifier OA1 is rectified and smoothed by a diode D1 and a capacitor C1, and then, inputted to a non-inverting input terminal of an operational amplifier OA2. In the case where the input voltage is larger than a threshold voltage Vthd determined by a constant-voltage source “V” and a variable resistor R5, a positive voltage is generated at the output terminal of the operational amplifier OA2. Thereby, the current value generated in the power receiving unit 27 can be measured. The output voltage of the operational amplifier OA2 is converted into a digital signal and supplied to the control unit (FIG. 2). Note that the resistance value of the resistor R1 is much smaller than the respective resistance values of the resistors R2, R3, R4, and R6.

Referring to FIG. 2 again, the control unit 22 determines whether or not the ultrasonic probe 1 is within a region where the energy wirelessly fed from the power feeding device 47 or the like can be received, based on the detection result of the received power detecting unit 28. Concurrently or instead thereof, by transmitting the detection result of the received power detecting unit 28 to the ultrasonic diagnostic apparatus main body 2 (FIG. 3), the control unit 42 of the ultrasonic diagnostic apparatus main body 2 may determine whether or not the ultrasonic probe 1 is within a region where the energy wirelessly fed from the power feeding device 47 or the like can be received.

The display control unit 29a controls the display unit 29b to display a warning, etc. under the control of the control unit 22. The display unit 29b includes a lighting device such as an LED or a display device such as an LCD, and displays a warning, etc. under the control of the display control unit 29a.

In the above-mentioned configuration, the transmission control unit 12, the reception control unit 14, the orthogonal detection processing unit 154 (FIG. 4), the sampling units 155a and 155b (FIG. 4), the parallel/serial conversion unit 16, the communication control unit 18, the control unit 22, the battery control unit 24, and the display control unit 29a may be formed of digital circuits, or formed of a CPU and software (programs) for allowing the CPU to perform various kinds of processing. The software (programs) is stored in the storage unit 23. Alternatively, the orthogonal detection processing unit 154 may be formed of analog circuits. In this case, the ADC 153 is omitted, and A/D conversion of the complex baseband signal is performed by the sampling units 155a and 155b.

On the other hand, referring to FIG. 3, the ultrasonic diagnostic apparatus main body 2 includes a wireless communication unit 31, a communication control unit 32, a serial/parallel conversion unit 33, an image forming unit 34, a display control unit 35, a display unit 36, an operation unit 41, a control unit 42, a storage unit 43, a power supply control unit 44, a power supply switch 45, a power supply unit 46, and a power feeding unit 47.

Here, the wireless communication unit 31 and the communication control unit 32 form a receiving unit for receiving the transfer signal and the detection signal representing the detection result of the received power detecting unit 28 and/or the determination result of the power receiving status detecting unit which signals are transmitted by the transmitting unit of the ultrasonic probe 1. Further, the serial/parallel conversion unit 33 and the image forming unit 34 form an image signal generating unit for generating an image signal based on the transfer signal received by the receiving unit.

The wireless communication unit 31 makes wireless communication between the ultrasonic probe 1 and itself, and thereby receives the transfer signal and the detection signal from the ultrasonic probe 1 and transmits various kinds of control signals to the ultrasonic probe 1. The wireless communication unit 31 demodulates the signals received by an antenna to output the detection signal and output serial sample data (the transfer signal) representing the complex baseband signals obtained from the reception signals outputted from the plural ultrasonic transducers.

The communication control unit 32 detects the detection signal outputted from the wireless communication unit 31 and outputs the detection signal to the control unit 42. The serial/parallel conversion unit 33 converts the serial sample data outputted from the wireless communication unit 31 into parallel sample data corresponding to the plural ultrasonic transducers.

The image forming unit 34 generates a B-mode image signal as tomographic image information on tissues within the object based on the parallel sample data outputted from the serial/parallel conversion unit 33. The image forming unit 34 includes a reception delay pattern storage unit 341, a phase matching and adding unit 342, a memory 343, and an image processing unit 344.

The reception delay pattern storage unit 341 stores plural reception delay patterns to be used when reception focusing processing is performed on the complex baseband signals obtained from the reception signals outputted from the plural ultrasonic transducers. The phase matching and adding unit 342 selects one reception delay pattern from among the plural reception delay patterns stored in the reception delay pattern storage unit 341 according to the reception direction set in the control unit 42, and performs reception focusing processing by providing delays to the complex baseband signals based on the selected reception delay pattern and adding the complex baseband signals to one another. By the reception focusing processing, baseband signals (sound ray signals), in which the focus of the ultrasonic echoes is narrowed, are formed.

The memory 343 sequentially stores the sound ray signals generated by the phase matching and adding unit 342. The image processing unit 344 generates a B-mode image signal as tomographic image information on tissues within the object based on the sound ray signals generated by the phase matching and adding unit 342 in the live mode and based on the sound ray signals stored in the memory 343 in the freeze mode.

The image processing unit 344 includes an STC (sensitivity time control) part, and a DSC (digital scan converter). The STC part performs attenuation correction on the sound ray signals by distance according to the depths of the reflection positions of ultrasonic waves. The DSC converts (raster-converts) the sound ray signals corrected by the STC part into an image signal that follows the normal scan system of television signals and performs necessary image processing such as gradation processing to generate the B-mode image signal.

The display control unit 35 allows the display unit 36 to display ultrasonic diagnostic images based on the B-mode image signal generated by the image forming unit 34. The display unit 36 includes a display device such as an LCD, and displays ultrasonic diagnostic images under the control of the display control unit 35.

The control unit 42 controls the respective units of the ultrasonic diagnostic apparatus according to the operation of an operator using the operation unit 41. The power supply switch 45 is provided in the ultrasonic diagnostic apparatus main body 2, and the power supply control unit 44 controls ON/OFF of the power supply unit 46 according to the status of the power supply switch 45. The power feeding unit 47 provided in a probe holder feeds power to the power receiving unit 27 of the ultrasonic probe 1 (FIG. 2) by the electromagnetic induction action using an LC resonance circuit.

Generally, the energy that can be wirelessly received attenuates as the distance between the power feeding unit 47 and the power receiving unit 27 becomes longer. In the embodiment, the power feeding unit 47 is built in a probe holder 48, and therefore, when the ultrasonic probe 1 is held in the probe holder 48, the power receiving unit 27 is positioned closely to the power feeding unit 47 and wireless power feed with the highest efficiency can be realized. However, this does not mean that the condition, in which the ultrasonic probe 1 is held in the probe holder 48, is an essential requirement for power feed. Although there is some attenuation depending on the distance, when the ultrasonic probe 1 exists within a certain degree of distance range from the power feeding unit 47, feed efficiency to some degree can be obtained. In the case where the other power feeding device 5 as shown in FIG. 1 is used for power feed, the other power feeding device 5 includes the same configurations as those of the wireless communication unit 31, the communication control unit 32, the control unit 42, the power supply control unit 44, the power supply unit 46, and the power feeding unit 47, and receives the detection signal in the same manner as described above and controls the power feeding unit 47.

In the above-mentioned configuration, the communication control unit 32, the serial/parallel conversion unit 33, the phase matching and adding unit 342, the image processing unit 344, the display control unit 35, the control unit 42, and the power supply control unit 44 are formed of a CPU and software (programs) for allowing the CPU to perform various kinds of processing. However, they may be formed of digital circuits. The software (programs) is stored in the storage unit 43. As a recording medium in the storage unit 43, not only a built-in hard disk but also a flexible disk, MO, MT, RAM, CD-ROM, DVD-ROM, or the like may be used.

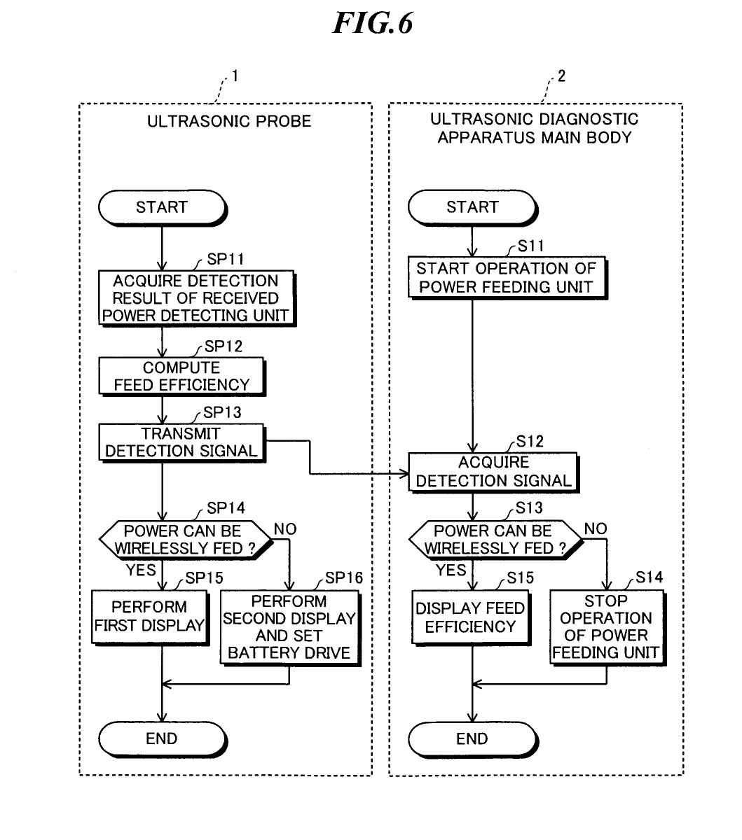

Next, an operation example of the ultrasonic diagnostic apparatus according to the first embodiment of the present invention will be explained by referring to FIGS. 2, 3 and 6. FIG. 6 is a flowchart for explanation of the operation example of the ultrasonic diagnostic apparatus according to the first embodiment of the present invention.

When a predetermined event occurs in the ultrasonic diagnostic apparatus main body 2, for example, the operator turns on the power supply switch 45 of the ultrasonic diagnostic apparatus main body 2, at step S11, the ultrasonic diagnostic apparatus main body 2 starts the operation of the power feeding unit 47 and waits until the detection signal is transmitted from the ultrasonic probe 1.

On the other hand, when a predetermined event occurs in the ultrasonic probe 1, for example, the operator turns on the power supply switch 25 of the ultrasonic probe 1, at step SP 11, the control unit 22 of the ultrasonic probe 1 acquires the detection result of the received power detecting unit 28. The detection result is acquired as a signal representing whether or not an induced electromotive force equal to or more than a predetermined value is generated in the LC circuit of the received power detecting unit 28 by the magnetic field generated in the power feeding unit 47.

After acquiring the detection result of the received power detecting unit 28, at step SP12, the control unit 22 computes the feed efficiency of the energy being received. As a computation procedure, for example, the feed efficiency is computed by computing a power value based on the current value obtained in the received power detecting unit 28 and the known resistance value of the load, and dividing the power value by the known consumed power value of the power feeding unit 47.

Then, the control unit 22 outputs data of the detection result (and the feed efficiency according to need) of the received power detecting unit 28 to the communication control unit 18. At step SP13, the communication control unit 18 controls the wireless communication unit 17, and the wireless communication unit 17 transmits the detection signal based on the data.

The control unit 42 of the ultrasonic diagnostic apparatus main body 2 waiting at step S11 acquires the detection signal received by the wireless communication unit 31 at step S12. Based on the detection signal, at step S13, the control unit 42 determines whether or not the ultrasonic probe 1 is within a region where the energy wirelessly fed from the power feeding unit 47 can be received. In the case where the ultrasonic probe 1 is not within a region where the energy wirelessly fed from the power feeding unit 47 can be received (step S13: NO), it is impossible to sufficiently feed power even by operating the power feeding unit 47, and therefore, the control unit 42 stops the operation of the power feeding unit 47 at step S14. It is preferable that whether or not the ultrasonic probe 1 is within a region where the energy wirelessly fed from the power feeding unit 47 can be received is determined by comparing the feed efficiency computed at step SP12 with a predetermined threshold value. Thereby, power feed with low feed efficiency can be avoided and wasteful power consumption can be suppressed.

In the case where the ultrasonic probe 1 is within a region where the energy wirelessly fed from the power feeding unit 47 can be received (step S13: YES), the control unit 42 controls the display control unit 35 at step S15 to allow the display unit 36 to display the feed efficiency to the ultrasonic probe 1 during power feed, for example.

Also in the ultrasonic probe 1 that has transmitted the detection signal, at step SP14, the control unit 22 determines whether or not the ultrasonic probe 1 is within a region where the energy wirelessly fed from the power feeding unit 47 can be received. The determination may be performed separately from the above-mentioned determination at step S13, or the determination result at step S13 may be received from the ultrasonic diagnostic apparatus main body 2 and the result may be used. Alternatively, in the opposite way, the determination result by the control unit 22 may be transmitted to the ultrasonic diagnostic apparatus main body 2 and the determination result by the control unit 22 may be used instead of the above-mentioned determination at step S13.

In the case where the ultrasonic probe 1 is within a region where the energy wirelessly fed from the power feeding unit 47 can be received (step SP14: YES), the control unit 22 controls the display control unit 29a at step SP15 to allow the display unit 29b to perform the first display. As the first display, for example, a green LED is lighted.

In the case where the ultrasonic probe 1 is not within a region where the energy wirelessly fed from the power feeding unit 47 can be received (step SP14: NO), the control unit 22 controls the display control unit 29a at step SP16 to allow the display unit 29b to perform the second display. As the second display, for example, a red LED is lighted. Further, the control unit 22 controls the battery control unit 24 to set such that only the battery 26 is used as a drive power supply of the ultrasonic probe 1.

In the above description, the case where the operation of the power feeding unit 47 is stopped when charging efficiency is less than a threshold value has been explained. However, the operation of the power feeding unit 47 may be continued when the charging efficiency is less than the threshold value, and the second display in the ultrasonic probe 1 may be performed and whether power is fed or not may be left up to a user.

Further, in the above description, the case where whether power can be wirelessly fed or not is determined by computing the feed efficiency of energy being received has been explained. However, whether power can be received or not may be determined by comparing the current value itself obtained by the received power detecting unit 28 with a predetermined threshold value.

Furthermore, in the above description, the case where whether power can be received or not is determined and the power feeding unit is operated or stopped with respect to one ultrasonic probe 1 has been explained. However, whether power can be received or not may be determined and the power feeding unit may be operated or stopped separately with respect to plural ultrasonic probes. In this case, IDs may be provided to the respective ultrasonic probes for identification of the plural ultrasonic probes, and the IDs may be included in the detection signal from the respective ultrasonic probes.

Moreover, in the above description, the case where the received power detecting unit 28 is connected to the LC circuit of the power receiving unit 27 has been explained. However, the power may be charged directly from the LC circuit of the power receiving unit 27 into the battery 26 not via the received power detecting unit 28, and an LC circuit may be provided in the received power detecting unit 28 separately from the LC circuit of the power receiving unit 27, and then, whether the power can be received or not in the power receiving unit 27 may be detected by sensing an output current of the LC circuit of the received power detecting unit 28.

Further, in the above description, the case where the transfer signal and the detection signal are transmitted by the same wireless communication unit 17 and received by the same wireless communication unit 31 has been explained. However, the signals may be transmitted or received by respective wireless communication units according to amounts or distances of the transfer.

Furthermore, in the above description, as a method of wirelessly feeding power, the case where the electric energy is converted into a magnetic field by using the LC resonance circuit and the magnetic field is reconverted into electric energy at the reception side has been explained. However, the electric energy may be converted into an electric field by using electrodes and the electric field may be reconverted into electric energy at the reception side. Alternatively, the electric energy may be converted into optical energy or thermal energy and transmitted to the reception side.

Next, an ultrasonic diagnostic apparatus according to the second embodiment of the present invention will be explained.

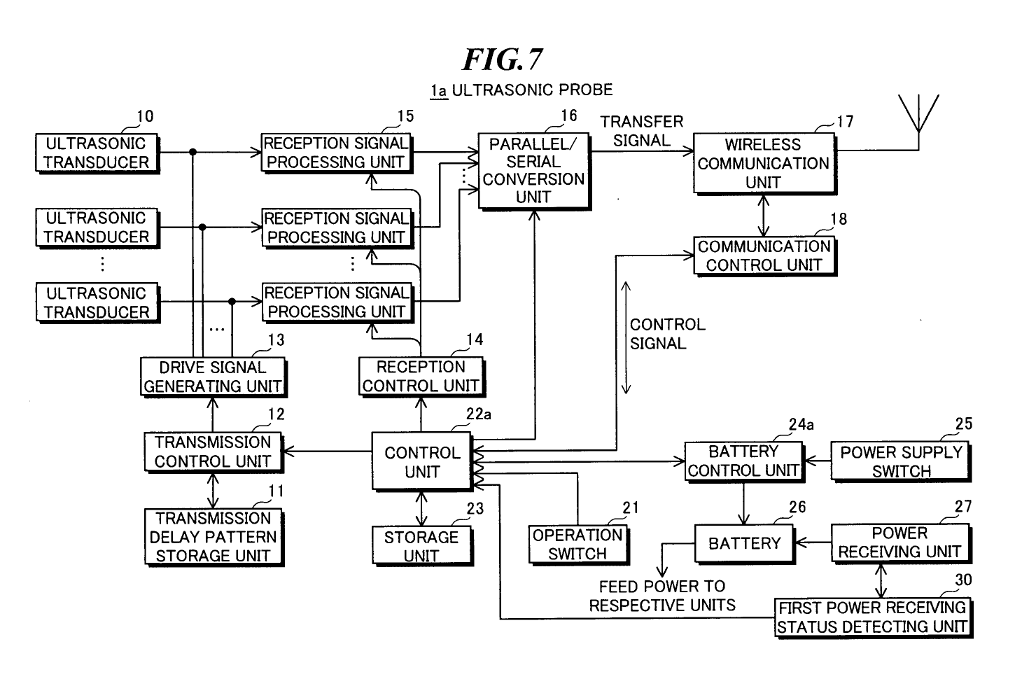

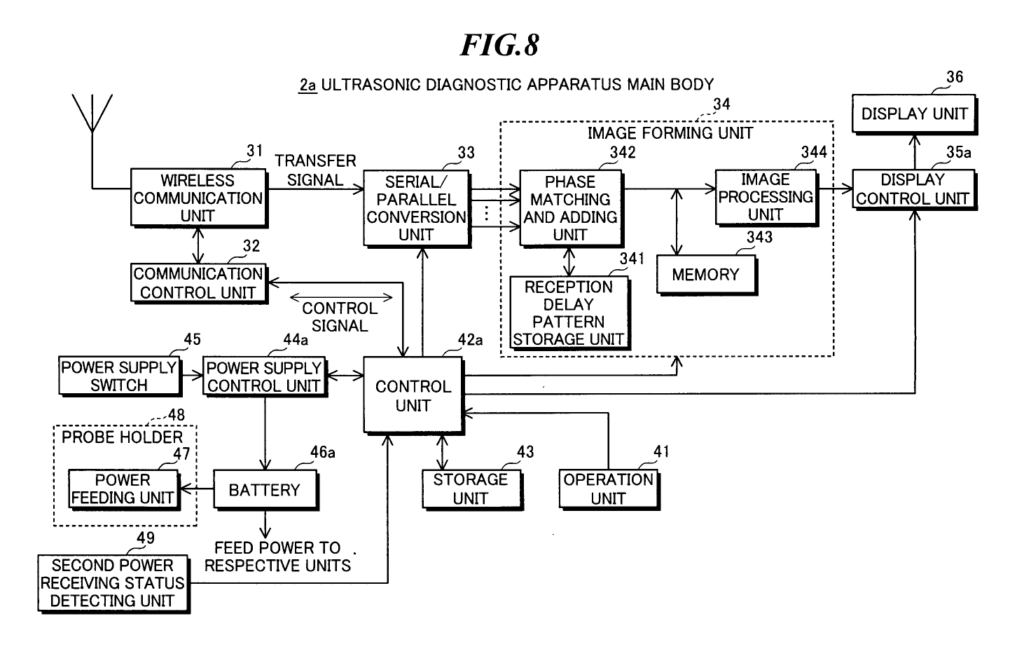

FIG. 7 is a block diagram showing a configuration of an ultrasonic probe according to the second embodiment of the present invention, and FIG. 8 is a block diagram showing a configuration of an ultrasonic diagnostic apparatus main body according to the second embodiment of the present invention. The ultrasonic probe 1a may be an external probe of linear-scan type, convex-scan type, sector-scan type, or the like, or an ultrasonic endoscopic probe of radial-scan type or the like.

As shown in FIG. 7, the ultrasonic probe 1a includes plural ultrasonic transducers 10 forming a one-dimensional or two-dimensional transducer array, a transmission delay pattern storage unit 11, a transmission control unit 12, a drive signal generating unit 13, a reception control unit 14, plural channels of reception signal processing units 15, a parallel/serial conversion unit 16, a wireless communication unit 17, a communication control unit 18, an operation switch 21, a control unit 22a, a storage unit 23, a battery control unit 24a, a power supply switch 25, a battery 26, a power receiving unit 27, and a first power receiving status detecting unit 30.

Here, the reception signal processing units 15 and the parallel/serial conversion unit 16 form a signal processing unit for performing signal processing on reception signals outputted from the plural ultrasonic transducers 10 to generate a transfer signal. The battery control unit 24a forms a remaining battery charge detecting unit for detecting remaining battery charge. The control unit 22a forms a feed time determining unit for determining a time, in which power can be fed from the battery, based on the remaining battery charge detected by the remaining battery charge detecting unit. The wireless communication unit 17 and the communication control unit 18 form a transmitting unit for transmitting the transfer signal generated by the signal processing unit and a detection signal representing a determination result of the first power receiving status detecting unit 30 and a determination result of the feed time determining unit. The power receiving unit 27 forms an energy conversion unit for converting energy wirelessly fed from the power feeding device into electric energy.

The plural ultrasonic transducers 10 transmit ultrasonic waves according to applied drive signals, and receive propagating ultrasonic echoes to output reception signals.

The transmission delay pattern storage unit 11 stores plural transmission delay patterns to be used when an ultrasonic beam is formed by using ultrasonic waves transmitted from the plural ultrasonic transducers 10. The transmission control unit 12 selects one transmission delay pattern from among the plural transmission delay patterns stored in the transmission delay pattern storage unit 11 according to a transmission direction set by the control unit 22a, and sets delay times to be respectively provided to the drive signals for the plural ultrasonic transducers 10 based on the selected transmission delay pattern. Alternatively, the transmission control unit 12 may set delay times such that the ultrasonic waves transmitted at a time from the plural ultrasonic transducers 10 reach the entire imaging region of the object.

The drive signal generating unit 13 includes plural pulsers, for example, and adjusts the amounts of delay of the drive signals such that the ultrasonic waves transmitted from the plural ultrasonic transducers 10 form an ultrasonic beam and supplies the drive signals to the plural ultrasonic transducers 10, or supplies the drive signals to the plural ultrasonic transducers 10 such that the ultrasonic waves transmitted at a time from the plural ultrasonic transducers 10 reach the entire imaging region of the object.

The reception control unit 14 controls the operation of the plural channels of reception signal processing units 15. Each channel of reception signal processing unit 15 performs orthogonal detection processing or orthogonal sampling processing on the reception signal outputted from the corresponding ultrasonic transducer 10 to generate a complex baseband signal, samples the complex baseband signal to generate sample data, and supplies the sample data to the parallel/serial conversion unit 16.

The parallel/serial conversion unit 16 converts the parallel sample data generated by the plural channels of reception signal processing units 15 into serial sample data (transfer signal). For example, the parallel/serial conversion unit 16 converts 128 channels of parallel data obtained based on the 64 reception signals outputted from the 64 ultrasonic transducers into one channel or two, three or four channels of serial sample data.

The wireless communication unit 17 modulates a carrier signal based on the transfer signal to generate a transmission signal and supplies the transmission signal to an antenna to transmit electric waves from the antenna so as to transmit the transfer signal. Further, the wireless communication unit 17 modulates a carrier signal based on the determination result of the first power receiving status detecting unit 30 and the determination result of the feed time by the control unit 22a to generate a transmission signal and supplies the transmission signal to the antenna to transmit electric waves from the antenna so as to transmit the detection signal.

In this manner, the wireless communication unit 17 performs wireless communication between the ultrasonic diagnostic apparatus main body 2a and itself, and thereby, transmits the transfer signal and the detection signal to the ultrasonic diagnostic apparatus main body 2a, and receives various kinds of control signals from the ultrasonic diagnostic apparatus main body 2a to output the received control signals to the communication control unit 18. The communication control unit 18 controls the wireless communication unit 17 such that the transfer signal and the detection signal are transmitted with transmission electric wave intensity set by the control unit 22a, and outputs the various kinds of control signals received by the wireless communication unit 17 to the control unit 22a. The control unit 22a controls the respective units of the ultrasonic probe 1a according to the various kinds of control signals transmitted from the ultrasonic diagnostic apparatus main body 2a.

The battery 26 supplies power to the respective units requiring power such as the drive signal generating unit 13, the reception signal processing units 15, the parallel/serial conversion unit 16, the wireless communication unit 17, the control unit 22a, and so on. The ultrasonic probe 1a is provided with the power supply switch 25, and the battery control unit 24a controls whether the power is supplied to the respective units from the battery 26 or not according to the status of the power supply switch 25. The battery 26 can be charged by using electric energy obtained by the power receiving unit 27 from the supplied energy.

The power receiving unit 27 is an electric circuit for converting the energy wirelessly supplied from the power feeding unit 47 of the ultrasonic diagnostic apparatus main body 2 (FIG. 8) or the other power feeding device 5 (FIG. 1) into electric energy, and thereby, receiving wirelessly fed power. The power receiving unit 27 generates an induced electromotive force from a magnetic field generated by the power feeding unit 47 by using an LC resonance circuit, for example.

The first power receiving status detecting unit 30 is an electric circuit for detecting an amount of the energy wirelessly fed from the power feeding unit 47 or another power feeding device 5, and thereby, determining whether or not the ultrasonic probe 1a is within a region where the energy wirelessly fed from the power feeding device 47 or the other power feeding device 5 can be received. The first power receiving status detecting unit 30 includes an LC circuit smaller than the LC circuit of the power receiving unit 27, and a current sensing circuit for sensing a current generated in the LC circuit due to the induced electromotive force, for example. The LC circuit of the first power receiving status detecting unit 30 is a small LC circuit so as to detect whether the power can be received or not even when the power feeding unit 47 is driven with low output.

Further, the battery control unit 24a detects a battery voltage, for example, to detect remaining battery charge. Generally, the higher the battery voltage, the higher the remaining battery charge, and parameters other than the battery voltage such as a temperature may be used for computation. The detection result of remaining battery charge by the battery control unit 24a is outputted to the control unit 22a. The control unit 22a acquires the detection result of remaining battery charge by the battery control unit 24a, and stores it as data showing a time-series change of remaining battery charge in the storage unit 23. Further, the control unit 22a computes an amount of change per time of remaining battery charge based on the time-series change of remaining battery charge stored in the storage unit 23. Then, the control unit 22a computes a remaining time, in which power can be fed from the battery, based on the remaining battery charge and the amount of change thereof per time.

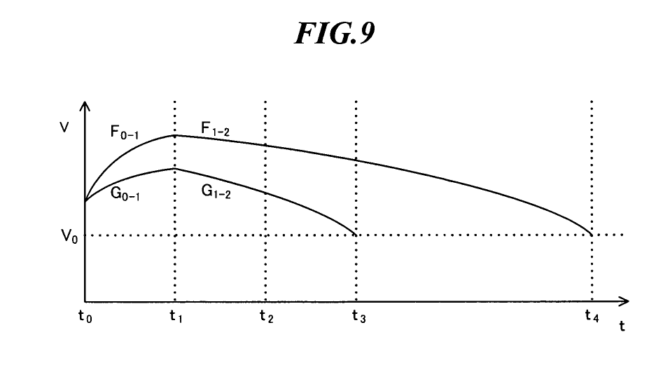

FIG. 9 is a graph showing a principle of computing a remaining time in which electric power can be supplied from a battery. The vertical axis of the graph indicates a battery voltage “V” and the horizontal axis indicates an elapsed time “t”.

In the case where charging is started at time t₀ and driving of the ultrasonic probe is started from time t₁, it is assumed that a driving time is computed at time t₁. From time t₀ to time t₁, the battery voltage “V” sharply rises as a curve F₀₋₁ when the charging efficiency is high, and the battery voltage “V” gently rises as a curve G₀₋₁ when the charging efficiency is low. Here, in the case where the driving of the ultrasonic probe is started from time t₁ while charging is continued, if the wireless charging can not keep up with the power consumption by the driving, the battery voltage “V” is expected to gently drop as a curve F₁₋₂ even when the charging efficiency is high. On the other hand, when the charging efficiency is low, the battery voltage “V” is expected to sharply drop as a curve G₁₋₂. Accordingly, from the gradient of the curve F₀₋₁ or G₀₋₁ showing the charging efficiency before driving and a known value showing consumed power during driving, time t₃ or t₄ when the battery voltage “V” reaches the lowest voltage V₀ at which the ultrasonic probe can be driven can be computed as the time in which power can be fed from the battery.

Further, in the case where the driving of the ultrasonic probe is started from time t₁ and time is elapsed to time t₂, the remaining time, in which power can be fed from the battery, may be computed at time t₂. In either case where the voltage “V” gently drops as the curve F₁₋₂ or the battery voltage “V” sharply drops as the curve G₁₋₂ from time t₁ to time t₂, time t₃ or t₄ when the battery voltage “V” reaches the lowest voltage V₀ at which the ultrasonic probe can be driven can be computed as the time, in which power can be fed from the battery, based on gradients of these curves and the battery voltage “V” at time t₂.

As described above, the time, in which power can be fed from the battery, may be computed based on the gradient of the curve of the battery voltage “V”. Alternatively, plural time-series change data of the battery voltage “V” in charging and discharging in the past are stored in the storage unit 23, and data near actual measurement values is extracted from the time-series change data, and thereby, the time, in which power can be fed from the battery, may be computed based on the time-series change data near actual measurement values.

In the above-mentioned configuration, the transmission control unit 12, the reception control unit 14, the parallel/serial conversion unit 16, the communication control unit 18, the control unit 22a, and the battery control unit 24a may be formed of digital circuits, or formed of a CPU and software (programs) for allowing the CPU to perform various kinds of processing. The software (programs) is stored in the storage unit 23.

On the other hand, referring to FIG. 8, the ultrasonic diagnostic apparatus main body 2a includes a wireless communication unit 31, a communication control unit 32, a serial/parallel conversion unit 33, an image forming unit 34, a display control unit 35a, a display unit 36, an operation unit 41, a control unit 42a, a storage unit 43, a power supply control unit 44a, a power supply switch 45, a battery 46a, the power feeding unit 47, and a second power receiving status detecting unit 49. Here, the wireless communication unit 31 and the communication control unit 32 form a receiving unit for receiving the transfer signal.

The wireless communication unit 31 makes wireless communication between the ultrasonic probe 1a and itself, and thereby receives the transfer signal and the detection signal from the ultrasonic probe 1a and transmits various kinds of control signals to the ultrasonic probe 1a. The wireless communication unit 31 demodulates the signal received by an antenna to output a detection signal and output serial sample data (the transfer signal) representing the complex baseband signals obtained from the reception signals outputted from the plural ultrasonic transducers.

The communication control unit 32 detects the detection signal outputted from the wireless communication unit 31 and outputs the detection signal to the control unit 42a. The serial/parallel conversion unit 33 converts the serial sample data outputted from the wireless communication unit 31 into parallel sample data corresponding to the plural ultrasonic transducers.

The image forming unit 34 generates a B-mode image signal as tomographic image information on tissues within the object based on the parallel sample data outputted from the serial/parallel conversion unit 33. The image forming unit 34 includes a reception delay pattern storage unit 341, a phase matching and adding unit 342, a memory 343, and an image processing unit 344.

The reception delay pattern storage unit 341 stores plural reception delay patterns to be used when reception focusing processing is performed on the complex baseband signals obtained from the reception signals outputted from the plural ultrasonic transducers. The phase matching and adding unit 342 selects one reception delay pattern from among the plural reception delay patterns stored in the reception delay pattern storage unit 341 according to the reception direction set in the control unit 42a, and performs reception focusing processing by providing delays to the complex baseband signals based on the selected reception delay pattern and adding the complex baseband signals to one another. By the reception focusing processing, baseband signals (sound ray signals) in which the focus of the ultrasonic echoes is narrowed are formed.

The memory 343 sequentially stores the sound ray signals generated by the phase matching and adding unit 342. The image processing unit 344 generates the B-mode image signal as tomographic image information on tissues within the object based on the sound ray signals generated by the phase matching and adding unit 342 in the live mode and based on the sound ray signals stored in the memory 343 in the freeze mode.

The image processing unit 344 includes an STC (sensitivity time control) part, and a DSC (digital scan converter). The STC part performs attenuation correction on the sound ray signals by distance according to the depths of the reflection positions of ultrasonic waves. The DSC converts (raster-converts) the sound ray signals corrected by the STC part into an image signal that follows the normal scan system of television signals and performs necessary image processing such as gradation processing to generate the B-mode image signal.

The display control unit 35a allows the display unit 36 to display an ultrasonic diagnostic image based on the B-mode image signal generated by the image forming unit 34. Further, the display control unit 35a allows the display unit 36 to display the determination result of the time, in which power can be fed from the battery, received from the ultrasonic probe 1a under the control of the control unit 42a. The display unit 36 includes a display device such as an LCD, and displays an ultrasonic diagnostic image and the time, in which power can be fed from the battery in ultrasonic probe 1a, under the control of the display control unit 35.

The control unit 42a controls the respective units of the ultrasonic diagnostic apparatus according to the operation of an operator using the operation unit 41. The ultrasonic diagnostic apparatus main body 2a is provided with the power supply switch 45, and the power supply control unit 44a controls ON/OFF of the power supply from the battery 46a according to the status of the power supply switch 45. The power feeding unit 47 provided in a probe holder 48 feeds power to the power receiving unit 27 of the ultrasonic probe 1a (FIG. 7) by the electromagnetic induction action using an LC resonance circuit.

The second power receiving status detecting unit 49 is an electric circuit for detecting an amount of the energy wirelessly fed from the other power feeding device 5 (FIG. 1), and thereby, determining whether or not the ultrasonic probe 1a is within a region where the energy wirelessly fed from the other power feeding device 5 can be received. The configuration of the second power receiving status detecting unit 49 is the same as that of the above-mentioned first power receiving status detecting unit 30 provided in the ultrasonic probe 1a, and the explanation thereof will be omitted.

In the above-mentioned configuration, the communication control unit 32, the serial/parallel conversion unit 33, the phase matching and adding unit 342, the image processing unit 344, the display control unit 35a, the control unit 42a, and the power supply control unit 44a are formed of a CPU and software (programs) for allowing the CPU to perform various kinds of processing, but they may be formed of digital circuits. The software (programs) is stored in the storage unit 43. As a recording medium in the storage unit 43, not only a built-in hard disk but also a flexible disk, MO, MT, RAM, CD-ROM, DVD-ROM, or the like may be used.

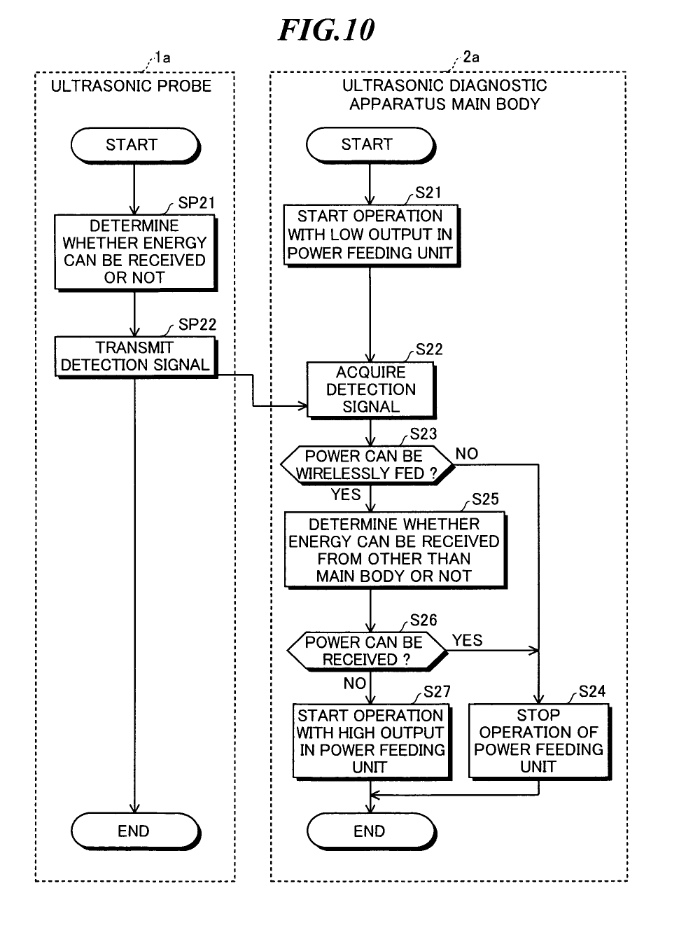

Next, an operation example of the ultrasonic diagnostic apparatus according to the second embodiment of the present invention will be explained by referring to FIGS. 1, 7, 8, and 10. FIG. 10 is a flowchart for explanation of an operation example of the ultrasonic diagnostic apparatus according to the second embodiment of the present invention.

When a predetermined event occurs in the ultrasonic diagnostic apparatus main body 2a, for example, the operator turns on the power supply switch 45 of the ultrasonic diagnostic apparatus main body 2a, at step S21, the control unit 42a of the ultrasonic diagnostic apparatus main body 2a starts the operation of the power feeding unit 47. At this stage, the power feeding unit 47 does not immediately feed power to the ultrasonic probe 1a, but starts the operation with low output for searching for an ultrasonic probe that can be fed with power and requires power feed, and waits until the detection signal is transmitted from the ultrasonic probe la.

On the other hand, when a predetermined event occurs in the ultrasonic probe 1a, for example, the operator turns on the power supply switch 25 of the ultrasonic probe 1a, at step SP21, the first power receiving status detecting unit 30 of the ultrasonic probe 1a determines whether or not the ultrasonic probe 1a is within a region where the energy wirelessly fed from the power feeding device 47 or the other power feeding device 5 can be received. The control unit 22a acquires the determination result of the first power receiving status detecting unit 30. The determination result is acquired as a signal representing whether or not an induced electromotive force equal to or more than a predetermined value is generated in the LC circuit of the first power receiving status detecting unit 30 due to the magnetic field generated by the power feeding unit 47 or other power feeding device 5.

After acquiring the determination result of the first power receiving status detecting unit 30, the control unit 22a outputs data of the determination result to the communication control unit 18. At step SP22, the communication control unit 18 controls the wireless communication unit 17 to transmit the detection signal based on the data.

The control unit 42a of the ultrasonic diagnostic apparatus main body 2a waiting at step S21 acquires the detection signal received by the wireless communication unit 31 at step S22. According to the detection signal, the control unit 42a determines whether the ultrasonic probe 1a can be wirelessly fed with power or not at step S23. In the case where the ultrasonic probe 1a cannot be wirelessly fed with power (step S23: NO), it is impossible to feed power even by operating the power feeding unit 47, and thus, the control unit 42a stops the operation of the power feeding unit 47 at step S24.

In the case where the ultrasonic probe 1a can be wirelessly fed with power (step S23: YES), at step S25, the second power receiving status detecting unit 49 determines whether or not the ultrasonic probe 1a is within a region where the energy wirelessly fed from the other power feeding device 5 than the ultrasonic diagnostic apparatus main body 2a can be received. The control unit 42a acquires the determination result of the second power receiving status detecting unit 49. The determination result is acquired as a signal representing whether or not an induced electromotive force equal to or more than a predetermined value is generated in the LC circuit of the second power receiving status detecting unit 49 due to the magnetic field generated by the other power feeding device 5. If there is a possibility that an interference occurs between the magnetic field generated by the power feeding unit 47 and the magnetic field generated by the other power feeding device 5, the operation of the power feeding unit 47 may be stopped only while the determination result of the second power receiving status detecting unit 49 is acquired, and thereby, the determination result only based on the magnetic field generated in the other power feeding device 5 can be acquired.

Then, at step S26, the control unit 42a determines whether or not the ultrasonic probe 1a can be wirelessly fed with power due to the magnetic field generated by the other power feeding device 5. In the case where the induced electromotive force equal to or more than the predetermined value has been generated in the LC circuit of the second power receiving status detecting unit 49 at step S25, it can be determined that the ultrasonic probe 1a can be wirelessly fed with power due to the magnetic field generated by the other power feeding device 5.

In the case where the ultrasonic probe 1a can be wirelessly fed with power due to the magnetic field generated by the other power feeding device 5 (step S26: YES), the control unit 42a stops the operation of the power feeding unit 47 at step S24. In this case, the ultrasonic probe 1a can be wirelessly fed with power due to the magnetic field generated by only the other power feeding device 5. As described above, by stopping the operation of the power feeding unit 47 when power can be fed from both the power feeding unit 47 and the other power feeding device 5, it is unnecessary for the ultrasonic diagnostic apparatus main body 2a to have a complicated configuration for controlling the other power feeding device 5, and the interference of wireless power feed can be avoided by a simple configuration. Further, draining of the battery 46a of the ultrasonic diagnostic apparatus main body 2a can be suppressed.

In the case where the ultrasonic probe 1a can not be wirelessly fed with power due to the magnetic field generated by the other power feeding device 5 (step S26: NO), the control unit 42a starts operation of the power feeding unit 47 with high output at step S27. Thereby, power is fed to the ultrasonic probe 1a that cannot be fed with power from the other power feeding device 5, and the examination can be continued.

In the above description, the case where the operation of the power feeding unit 47 is stopped when power can be fed from both the power feeding unit 47 and the other power feeding device 5 has been explained. However, contrary, a priority may be given to the power feed from the power feeding unit 47, and the operation of the other power feeding device 5 may be stopped.

Further, in the above description, the case where whether power can be fed from the power feeding unit 47 or not and whether power can be fed from the other power feeding device 5 or not are determined by the first power receiving status detecting unit 30 provided in the ultrasonic probe 1a and the second power receiving status detecting unit 49 provided in the ultrasonic diagnostic apparatus main body 2a has been explained. However, the power receiving status detecting unit may be provided in another device. Alternatively, the power receiving status detecting unit may be provided only in the ultrasonic probe 1a, and thereby, from which power feeding unit power can be fed may be determined by shifting times of the power feed from the power feeding unit 47 and the power feed from the other power feeding device 5.

Furthermore, in the above description, the case where whether power can be received or not is determined and the power feeding unit is operated or stopped with respect to one ultrasonic probe 1a has been explained. However, whether power can be received or not may be determined and the power feeding unit may be operated or stopped separately with respect to plural ultrasonic probes. In this case, IDs may be provided to the respective ultrasonic probes for identification of the plural ultrasonic probes, and the IDs may be included in the detection signals from the respective ultrasonic probes.

Moreover, in the above description, the case where the LC circuit of the first power receiving status detecting unit 30 is provided separately from the LC circuit of the power receiving unit 27 has been explained. However, whether the power can be received or not in the power receiving unit 27 may be detected by sensing the output current of the LC circuit of the power receiving unit 27 and so on.

Further, in the above description, the case where the transfer signal and the detection signal are transmitted by the same wireless communication unit 17 and received by the same wireless communication unit 31 has been explained. However, the signals may be transmitted or received by respective wireless communication units according to the amounts or distances of transfer.

Furthermore, in the above description, as a method of wirelessly feeding power, the case where the electric energy is converted into the magnetic field by using the LC resonance circuit and the magnetic field is reconverted into electric energy at the reception side has been explained. However, the electric energy may be converted into an electric field by using electrodes and the electric field may be reconverted into electric energy at the reception side. Alternatively, the electric energy may be converted into optical energy or thermal energy and transmitted to the reception side.

1. An ultrasonic probe comprising:
plural ultrasonic transducers for transmitting ultrasonic waves according to drive signals, and receiving ultrasonic echoes to output reception signals;
a signal processing unit for performing signal processing on the reception signals outputted from said plural ultrasonic transducers to generate a transfer signal;
an energy conversion unit for converting energy wirelessly fed from a power feeding device into electric energy;
a power receiving status detecting unit for detecting an amount of the energy wirelessly fed from said power feeding device, and determining whether or not said ultrasonic probe is within a region where the energy wirelessly fed from said power feeding device can be received; and
a transmitting unit for transmitting a determination result of said power receiving status detecting unit to said power feeding device.

2. The ultrasonic probe according to claim 1, wherein said power receiving status detecting unit computes feed efficiency of the energy wirelessly fed from said power feeding device.

3. The ultrasonic probe according to claim 2, wherein said power receiving status detecting unit determines whether or not the feed efficiency of the energy wirelessly fed from said power feeding device is equal to or more than a threshold value, and thereby, determines whether or not said ultrasonic probe is within the region where the energy wirelessly fed from said power feeding device can be received.

4. The ultrasonic probe according to claim 1, further comprising:
a display unit for performing first display in the case where said power receiving status detecting unit determines that said ultrasonic probe is within the region where the energy wirelessly fed from said power feeding device can be received.

5. The ultrasonic probe according to claim 4, wherein said display unit performs second display in the case where said power receiving status detecting unit determines that said ultrasonic probe is not within the region where the energy wirelessly fed from said power feeding device can be received.

6. The ultrasonic probe according to claim 1, further comprising:
a battery for accumulating the electric energy converted by said energy conversion unit; and
a power supply selecting unit for selecting the battery as a drive power supply in the case where said power receiving status detecting unit determines that said ultrasonic probe is not within the region where the energy wirelessly fed from said power feeding device can be received.

7. An ultrasonic probe comprising:
plural ultrasonic transducers for transmitting ultrasonic waves according to drive signals, and receiving ultrasonic echoes to output reception signals;
a signal processing unit for performing signal processing on the reception signals outputted from said plural ultrasonic transducers to generate a transfer signal;
an energy conversion unit for converting energy wirelessly fed from a power feeding device into electric energy;
a battery for accumulating the electric energy converted by said energy conversion unit and supplying electric power to at least said signal processing unit;
a remaining battery charge detecting unit for detecting remaining battery charge of said battery; and
a feed time determining unit for determining a time, in which electric power can be supplied from said battery, based on the remaining battery charge detected by said remaining battery charge detecting unit.

8. The ultrasonic probe according to claim 7, wherein said feed time determining unit determines the time, in which electric power can be supplied from said battery, based on the remaining battery charge detected by said remaining battery charge detecting unit and an amount of change per time of said remaining battery charge.

9. The ultrasonic probe according to claim 7, further comprising:
a transmitting unit for transmitting a determination result of said feed time determining unit to an outside.

10. The ultrasonic probe according to claim 7, further comprising:
a power receiving status detecting unit for detecting an amount of the energy wirelessly fed from said power feeding device, and determining whether or not said ultrasonic probe is within a region where the energy wirelessly fed from said power feeding device can be received.

11. The ultrasonic probe according to claim 10, further comprising:
a transmitting unit for transmitting a determination result of said power receiving status detecting unit to said power feeding device.

12. An ultrasonic diagnostic apparatus comprising an ultrasonic probe and an ultrasonic diagnostic apparatus main body;
said ultrasonic probe including:
plural ultrasonic transducers for transmitting ultrasonic waves according to drive signals, and receiving ultrasonic echoes to output reception signals;
a signal processing unit for performing signal processing on the reception signals outputted from said plural ultrasonic transducers to generate a transfer signal;
an energy conversion unit for converting energy wirelessly fed from said ultrasonic diagnostic apparatus main body into electric energy;
a received power detecting unit for detecting an amount of the energy wirelessly fed from said ultrasonic diagnostic apparatus main body; and
a transmitting unit for transmitting the transfer signal generated by said signal processing unit and a detection result of said received power detecting unit to said ultrasonic diagnostic apparatus main body; and
said ultrasonic diagnostic apparatus main body including:
a receiving unit for receiving the transfer signal and the detection result of said received power detecting unit transmitted by said transmitting unit;
an image signal generating unit for generating an image signal based on the transfer signal received by said receiving unit;
a power feeding unit for wirelessly feeding energy to said ultrasonic probe; and
a control unit for determining whether or not said ultrasonic probe is within a region where the energy wirelessly fed from said power feeding unit can be received, based on the detection result of said received power detecting unit received by said receiving unit.

13. The ultrasonic diagnostic apparatus according to claim 12, wherein said control unit computes feed efficiency of the energy wirelessly fed from said power feeding unit, based on the detection result of said received power detecting unit received by said receiving unit.

14. The ultrasonic diagnostic apparatus according to claim 13, wherein said control unit determines whether or not the feed efficiency of the energy wirelessly fed from said power feeding unit is equal to or more than a threshold value, and thereby, determines whether or not said ultrasonic probe is within the region where the energy wirelessly fed from said power feeding unit can be received.

15. The ultrasonic diagnostic apparatus according to claim 12, wherein said control unit controls said power feeding unit to stop feed of the energy in the case of determining that said ultrasonic probe is not within the region where the energy wirelessly fed from said power feeding unit can be received.

16. An ultrasonic diagnostic apparatus comprising an ultrasonic probe and an ultrasonic diagnostic apparatus main body;
said ultrasonic probe including:
plural ultrasonic transducers for transmitting ultrasonic waves according to drive signals, and receiving ultrasonic echoes to output reception signals;
a signal processing unit for performing signal processing on the reception signals outputted from said plural ultrasonic transducers to generate a transfer signal;
an energy conversion unit for converting energy wirelessly fed from at least one of said ultrasonic diagnostic apparatus main body and another power feeding device into electric energy;
a battery for accumulating the electric energy converted by said energy conversion unit and supplying electric power to at least said signal processing unit;
a remaining battery charge detecting unit for detecting remaining battery charge of said battery;
a feed time determining unit for determining a time, in which electric power can be supplied from said battery, based on the remaining battery charge detected by said remaining battery charge detecting unit; and
a transmitting unit for transmitting the transfer signal generated by said signal processing unit and a determination result of said feed time determining unit to said ultrasonic diagnostic apparatus main body; and
said ultrasonic diagnostic apparatus main body including:
a receiving unit for receiving the transfer signal generated by said signal processing unit and the determination result of said feed time determining unit;
an image signal generating unit for generating an image signal based on the transfer signal received by said receiving unit;
a display unit for displaying the determination result of said feed time determining unit; and
a power feeding unit for wirelessly feeding energy to said ultrasonic probe.

17. The ultrasonic diagnostic apparatus according to claim 16, wherein:
said ultrasonic probe further includes a power receiving status detecting unit for detecting an amount of the energy wirelessly fed from at least one of said ultrasonic diagnostic apparatus main body and said other power feeding device, and determining whether or not said ultrasonic probe is within a region where the energy wirelessly fed from at least one of said ultrasonic diagnostic apparatus main body and said other power feeding device can be received; and
said ultrasonic diagnostic apparatus main body further includes a control unit for controlling said power feeding unit to stop feed of the energy in the case where said power receiving status detecting unit determines that said ultrasonic probe is within the region where the energy wirelessly fed from at least one of said ultrasonic diagnostic apparatus main body and said other power feeding device can be received.

18. The ultrasonic diagnostic apparatus according to claim 17, wherein:
said ultrasonic diagnostic apparatus main body further includes a second power receiving status detecting unit for detecting an amount of the energy wirelessly fed from said other power feeding device, and determining whether or not said ultrasonic probe is within a region where the energy wirelessly fed from said other power feeding device can be received; and
said control unit controls said power feeding unit to stop feed of the energy in the case where said second power receiving status detecting unit determines that said ultrasonic probe is within the region where the energy fed from said other power feeding device can be received.
